(19) United States
(12) Patent Application Publication
(10) Pub. No.: US 2025/0345163 A1
(43) Pub. Date: Nov. 13, 2025

(54) DISPOSABLE SINGLE-USE MEDICAMENT DISPENSER

(71) Applicant: PharmaPhDII, LLC., Plantation, FL (US)

(72) Inventor: Max Arocha, Plantation, FL (US)

(73) Assignee: PharmaPhDII, LLC., Plantation, FL (US)

(21) Appl. No.: 18/661,593

(22) Filed: May 11, 2024

(51) Int. Cl. A61C 19/06; A61M 5/31; A61M 5/50

(52) U.S. Cl. CPC A61C 19/063; A61M 5/502; A61M 2005/3139

(57) ABSTRACT

The present invention relates to a single-use medicament dispenser or syringe, and a method of using the syringe to deliver a medicament. More specifically, the present invention relates to a single-use syringe that includes a mechanism for locking the syringe in a dispensed position following administration of a medicament therefrom, and thereby preventing reuse of the syringe. A system or kit comprising a plurality of single-use syringes according to the invention can be provided, packaged together, with written instructions for use.

### BACKGROUND OF THE INVENTION

Periodontitis is a bacterial infection of the oral tissues that retain the teeth in the jawbone. Untreated, periodontitis can damage the ligaments and bone, and can cause enlargement of the natural pockets between the teeth and periodontal tissue (gums), and result in loosening or even loss of teeth held by the surrounding infected tissue.

Currently, preferred methods of treating periodontal disease involve delivering therapeutic agents, such as antibiotics, directly to and within these periodontal pockets. However, arriving at methods and delivery devices that provide successful administration of antibiotic or other therapeutic agents to the periodontal pocket has proven to be difficult.

The development of microparticles in dry form has improved the delivery of antibiotic agents to the periodontal pocket. Devices have also been developed for delivery and administration of these microparticles. For example, U.S. Pat. Nos. 5,236,355, 5,500,228, and 5,622,498 describe a syringe having a tip which contains the microparticulate therapeutic agent, wherein the tip is adapted for delivery of microparticles to the periodontal pocket, and a plunger which forces the therapeutic agent from the tip and into the periodontal pocket. However, these devices were disadvantageous for delivering an accurate and consistent dose of the antibiotic.

Later improvements to these devices are described in U.S. Pat. Nos. 6,682,348 and 7,699,609 having a removable tip comprising the plunger, wherein the tip is fitted onto a specially adapted syringe-like actuator. However, these actuators can be difficult to manipulate wherein proper use for administration is an acquired skill, even by experienced professionals.

In addition, re-use of the actuators or delivery tips without proper cleaning and sterilization can present a substantial risk of infection to the patient. Mere exposure of a sterile tip or actuator to an unsterile environment can lead to contamination. For this reason, among others, syringes and other actuators, and hypodermic needles or other delivery tips used with those syringes or actuators, are intended for a single use only. One way to ensure single-use only is to disable the syringe or actuator such that re-use is prevented.

Syringes designed for preventing reuse exist. For example, certain syringe designs now include a needle capable of automatically retracting into the syringe barrel upon use. These syringes rely on either compression springs or gas powered, pneumatic mechanisms to retract the needle. Although effective in preventing syringe reuse, retractable mechanisms are expensive to mass produce.

Non-retractable mechanisms that can automatically disable a syringe are available. However, these mechanisms are complex and have too many parts to mass produce in a reliable, cost-effective way. Less expensive alternatives exist and include syringes with manually operated disabling mechanisms. However, manually disabling a syringe can prove to be unreliable since users may forget to disable the syringe. Even healthcare professions can fail to actively disable such syringes after use. A 2017 survey of healthcare professionals by the CDC found that syringe reuse can take place in the healthcare field with 12% of physicians and 3% of nurses admitting to having reused syringes in the workplace.

Accordingly, what is needed in the art is a simple, single-use syringe or actuator having an automatically disabling mechanism that prevents re-use of the syringe following the first, single use thereof. Such device that can be inexpensive to manufacture, easy to use, and disposable would be a further advancement to the art. These and other needs are met by the subject invention described herein, which can provide certain advantages heretofore unrealized.

### SUMMARY OF THE INVENTION

The present invention is directed to a single-use syringe, and a method of using said single-use syringe. In a preferred embodiment, the syringe and method of the invention are useful for delivering powdered medicament, e.g., microparticulate minocycline, to a periodontal pocket for treating or preventing periodontitis. Advantageously, the syringe of the invention can be disposed following use thereof.

The single-use syringe according to the subject invention comprises (a) a syringe body and (b) a locking plunger, wherein the locking plunger can be manually actuated into locked position, preventing reversal of the plunger, and preventing re-use of the syringe. The single-use syringe of the invention is made to be completely disposable following the use thereof.

The syringe body is hollow, comprising a first open end, a second open end, and a sidewall bounding a chamber formed within the syringe body. The syringe body can include a delivery tip for holding medicament, wherein the delivery tip comprises an exit port for expelling medicament and delivering medicament to the periodontal pocket of the patient in need thereof. The delivery tip is preferably formed integrally with the syringe body, e.g., the syringe body and delivery tip are formed as a single unit, or the delivery tip can be provided separate from the syringe body and be affixable to the syringe body for use. In a preferred embodiment, the delivery tip is configured at its exit port end as a dual-pronged beveled wedge. The chamber of the syringe of the subject invention does not contain or hold medicament. Rather, medicament is loaded into the delivery tip.

The plunger comprises a plunger stem portion which is slidingly disposed within the syringe body, i.e., within the chamber, and a plunger tip portion which is slidingly disposed within the delivery tip. In a preferred embodiment, the plunger stem can include a pawl for limiting movement of the plunger in one direction only. The inner surface of the sidewall of the syringe body can include at least one slot for matingly engaging the pawl and holding the plunger stem in a locked position. In a preferred embodiment, the inner surface of the sidewall of the syringe comprises two slots for matingly engaging the pawl. The at least one slot or two-slots can be formed partially through the sidewall or can extend completely through the sidewall of the syringe body.

In a two-slot embodiment, a first slot is formed to position the plunger in a primed position, wherein the plunger tip is proximate to the medicament, prior to administration of the medicament. A second slot is formed to position the plunger in a disabled position when fully actuated, wherein the plunger tip is moved to the end of the delivery tip to fully expel the medicament from the exit port and to the patient. When the plunger has been fully actuated and is in the locked position, the syringe is disabled, preventing further use of the syringe. Preferably, the plunger has at one end, opposite the plunger tip, a perpendicularly disposed thumb flange, serving to facilitate actuation of the plunger by the user and further providing a stop so the plunger does not completely enter the syringe chamber when fully actuated for delivery of medicament.

The delivery tip can be formed as a generally elongate tube extending from, and narrower in circumference than, the syringe body, forming two distinct portions of the syringe. In a first embodiment, the delivery tip is integrally formed as part of the syringe, having an internal bore extending throughout the length of the syringe, i.e., from the first open end to the second open end which terminates as the exit port from which medicament is expelled. The internal bore of the syringe body (the chamber portion) and the internal bore of the delivery tip are different diameters but are preferably cross-sectionally circular. In another embodiment, the delivery tip can be separately formed and affixable to the syringe body. Whether integrally formed as part of the syringe body or separately formed and affixable to the syringe body, the delivery tip can be formed at a fixed angle, or can be made of flexible material allowing the tip to be configured at a preferred angle for delivery of medicament.

The inner surface of the sidewall can also include a longitudinally disposed groove. The groove is configured to matingly receive a ridge provided on the plunger stem so that when the plunger stem is depressed, the groove and ridge engage for guiding the plunger stem and preventing rotation of the plunger within the chamber of the syringe.

Depression of the plunger stem to a disabled, locked position, also depresses the plunger tip which in turn expels the medicament bolus from the exit port of the delivery tip. The first end can also function as plunger stop to stop plunger stem from being depressed further when the syringe has been fully actuated. The single-use syringe can then be disposed of once the plunger stem is in a locked position and the syringe has been disabled.

The syringe body can further include a finger flange perpendicularly disposed onto or engaged with the syringe body to assist in the manipulation of the syringe by the user. In one embodiment, the finger flange can comprise a sleeve portion which engages the outer circumference of the syringe body and configured to permit the finger flange to rotate relative to the syringe body. The finger flange can also cover the at least one slot of the sidewall so that the plunger stem cannot be moved from the disabled, locked position into the primed, unlocked position.

A method for using the single-use syringe of the invention includes delivering a powdered medicament bolus to a periodontal pocket of a patient in need thereof, e.g., a patient having periodontitis. The single-use syringe of the invention is provided having a syringe body comprising at least one slot on an inner surface of the syringe body, a delivery tip, and plunger. The delivery tip is loaded with a medicament bolus. The medicament-loaded delivery tip and syringe body are positioned in a target periodontal pocket of a patient and the plunger is actuated such that the pawl engages the at least one slot, where the plunger is moved to the disabled position.

In a preferred method, the delivery tip is integrally formed as part of the syringe body having two slots, and is provided pre-loaded with medicament bolus. The plunger is provided in a primed position, i.e., wherein the pawl is engaged with the first slot. Following positioning of the delivery tip in a target periodontal pocket of a patient, the plunger can be actuated such that pawl is moved to engage the second slot, whereby the syringe is in a disabled position following delivery of medicament.

Following use of the single-use syringe, the syringe and delivery tip can be disposed into a trash receptacle to prevent re-use of the syringe.

### BRIEF DESCRIPTION OF THE DRAWINGS

The drawings as provided herein set forth exemplary embodiments of the present invention, the detailed description of which follows hereinbelow. The drawings are merely exemplary and are not intended to limit the invention as encompassed by the claims appended herewith.

FIG. 1 is an exploded view of the single-use syringe in accordance with an embodiment of the present invention.

FIG. 2A is a cross-sectional view of the single-use syringe of FIG. 1 in a primed, unlocked position.

FIG. 2B is a cross-sectional view of the single-use syringe of FIG. 1 in a disabled, locked position.

FIG. 3A is a side view of a delivery tip of the single-use syringe of FIG. 1.

FIG. 3B is a side view of the plunger tip of the single-use syringe of FIG. 1.

### DETAILED DESCRIPTION

The present invention is directed to a single-use syringe and a method of use thereof. The single-use syringe and method of the present invention is advantageously convenient, easy to operate, and safe to dispose. The subject single-use syringe and its components can be readily understood by reference to the description and drawings provided herein.

In a preferred embodiment, the single-use syringe is disposable and comprises a hollow syringe body which is formed by a sidewall having an outer surface, and an inner surface bounding a chamber formed within the syringe body. The inner surface of the syringe body has at least one slot for matingly receiving and engaging a pawl.

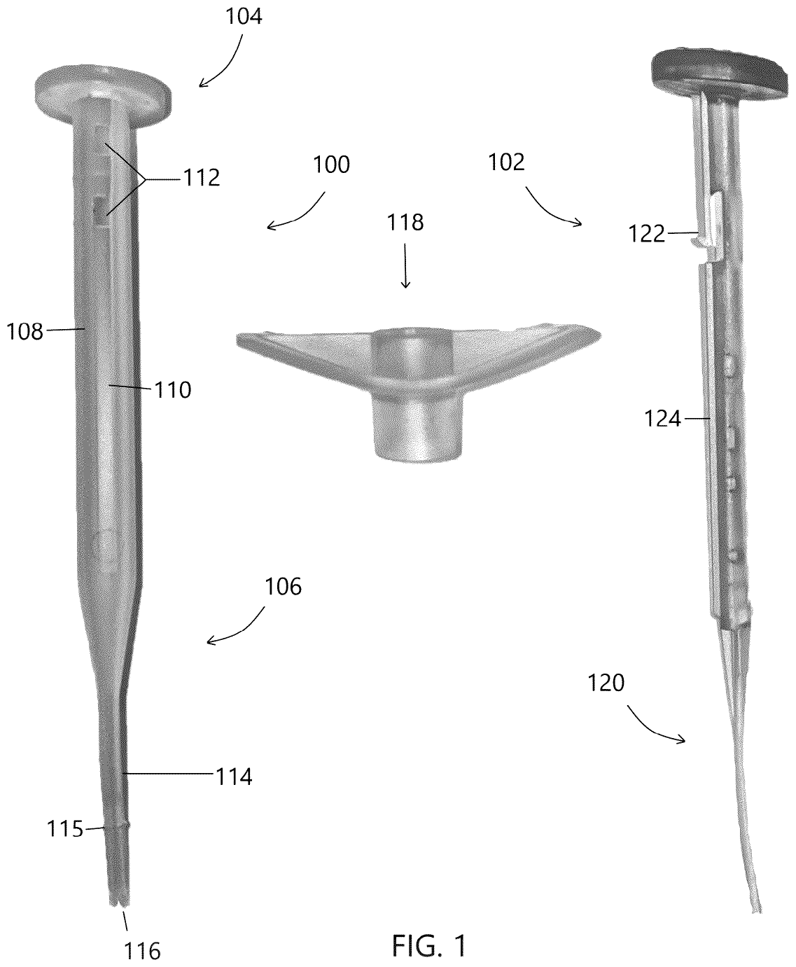

Referring to the Figures, FIG. 1 shows the components or an embodiment of the present invention, namely, syringe body 100, plunger 102, and finger flange 118.

The syringe body is generally elongate, having a first open end for receiving a plunger and a second open end comprising a delivery tip having a bore therethrough and being configured to hold and deliver a powdered medicament bolus. The delivery tip can be separate from and affixable to the syringe body, but is preferably integrally formed as part of the syringe body, wherein the syringe body tapers to a narrowing tip configuration.

Syringe body 100 is illustrated comprising a first open end 104, a second open end 106, and a sidewall 108 having an outer and inner surface bounding a chamber therewithin. The first end can also include a flange XX, serving as a plunger stop to stop plunger from being depressed further when the syringe has been fully actuated. The sidewall 108 has at least one slot 112, and preferably at least two slots formed therein, and a groove 110 on its inner surface.

In an embodiment where the syringe body and delivery tip are integrally formed as a single unit, the plunger can further comprise a delivery portion extending from the stem portion, and which is slidably disposable within the delivery tip. Thus, the plunger stem portion and delivery portion generally conform to the size and shape of the respective syringe body chamber and delivery tip bore in which they are disposed.

In one preferred embodiment, the pawl is integrally formed in the plunger stem portion, e.g., molded or die-cut as part of the plunger stem portion. The pawl permits movement of the plunger in one linear direction only, engaging the one or more slots and preventing movement in an opposite direction once engaged.

The syringe body can further include finger flange 118 to assist in the manipulation of the syringe. In certain embodiments, the finger flange can be configured to rotate so that a user can adjust their grip on the syringe. The finger flange can also cover the at least one slot of the sidewall so that the plunger stem cannot be moved from the disabled, locked position into the primed, unlocked position.

The syringe of the invention further comprises a plunger which has a stem portion that slidably fits within and engages the chamber. The plunger stem preferably has a capped distal end which stops movement of the plunger at the first open end of the syringe body. The plunger stem further comprises a pawl capable of matingly engaging the at least one slot disposed on the inner surface of the syringe body.

Plunger 102 is illustrated having a plunger stem portion 121 and plunger tip portion 120. Toward a distal end of the plunger stem portion is provided a pawl 122 for matingly engaging with the slot or slots 112 of syringe body 108. Plunger 102 can further comprise a longitudinally disposed ridge 124, which matingly engages with groove 110 formed in the syringe body. Plunger 102 can slidingly enter syringe body 108 through the first open end of the syringe body, where it can slidingly move within the chamber.

Groove 110 on the inner surface of the sidewall 108 matingly engages with ridge 124 and guides the plunger stem 102 as it slides into the syringe body 100. The pawl 122 engages with the at least one slot 112 of the sidewall 108 and holds the plunger stem 102 in a locked position upon actuation.

As will be further appreciated from the description herein, the delivery tip can be formed integrally with the syringe body or can be formed separate from the syringe body and removably attached thereto. In addition to holding a medicament bolus, a separable delivery tip can also house a plunger tip. The plunger tip is proximal to and abuts the medicament bolus such that it is operable to expel the medicament bolus out of the delivery tip upon actuation. In one embodiment of the present invention, the medicament bolus can be a pellet comprising dry particles, preferably microparticles, such as micronized minocycline.

The delivery tip can be configured as a generally elongate tube having an outer circumference and an internal bore disposed throughout its length and terminating in a medicament exit port. The internal bore can be substantially round at a proximal end, tapering to an elliptical circumference at a distal end.

Depression of the plunger stem from a primed, unlocked position to a disabled, locked position, also depresses the plunger tip which in turn expels the medicament bolus through a medicament exit port 116. Advantageously, the single-use syringe can then be disposed following use, wherein the plunger stem is in a locked position and the syringe has been disabled.

The syringe embodiment shown on FIG. 1 also illustrates a second end having a delivery tip 114 integrally formed with the syringe body 100. In addition to housing the plunger tip 120, the delivery tip 114 is configured to hold a medicament bolus which is proximal to and abuts the plunger tip 120. During actuation, the plunger stem 102 is depressed and in turn so is the plunger tip 120. As a result, the plunger tip 120 expresses the medicament bolus through the medicament exit port 116.

In a preferred embodiment, the delivery tip is angled to facilitate delivery of the medicament to the periodontal pocket by a user of the syringe. Thus, pushing of the plunger from a convenient manual position by the user can deliver the medicament at a different angle which is conducive to administering the medicament within a periodontal pocket. Typically, a preferred is angle is between about 10° and about 90° relative to a longitudinal axis of the syringe body. More preferably, the delivery tip is angled between about 15° and about 45° relative to a longitudinal axis of the syringe body. A more preferred angle of the delivery tip is about 30° relative to a longitudinal axis of the syringe body. However, the specific angle between about 10° and about 90° is not critical and can be varied according to the preference of the user.

The delivery tip can be marked, etched, or molded to comprise designations for measurement or calibration of the depth at which the delivery tip is positioned within the periodontal pocket. In one preferred embodiment, the calibration or measurement mark can comprise a raised ridge, which can also serve as a cap stop for a cap provided to cover the delivery tip.

The second open end of the delivery tip serves as an exit port for the medicament disposed therein. The exit port is preferably beveled so that the end is not blunted. A blunt end of the delivery tip can interfere with placement of the tip within the periodontal pocket. A more preferred delivery tip has a beveled end serving as the exit port wherein the bevel is configured as a two-pronged beveled wedge. The wedge shape can facilitate placement of the delivery tip within the periodontal pocket and can further facilitate expelling the medicament from the delivery tip. For example, the two-pronged beveled wedge configuration exposes the medicament on two sides. Adhering forces between the medicament and the tissue of the patient can case the removal of the medicament from the delivery tip and retention of the medicament where administered.

More preferably, the delivery tip is an elongate and tapered tube having an internal bore therethrough, wherein the bore is cross-sectionally circular. The cross-sectionally circular can be advantageous for measurement and dosing of the medicament held therewithin. However, it is preferred that the outer circumference of the delivery tip is cross-sectionally elliptical in shape. This elliptical outer circumference of the delivery tip can supplement the formation of the wedge shape of the delivery tip and can facilitate placement of the delivery tip relative to the periodontal pocket.

To guide the plunger stem position such that the pawl aligns with the one or more slots in the syringe body, a longitudinal ridge can be provided at least partially along at last one side of the plunger stem. In a preferred embodiment, this ridge will matingly engage with a groove formed on the inner surface of syringe body sidewall. The guiding ridge on the plunger stem and the groove formed on the inner surface of the syringe body sidewall further prevent rotation of the plunger stem within the chamber formed in and bounded by the syringe body.

In one preferred embodiment, the inner surface of the syringe body sidewall comprises two slots for positioning the plunger in a first or “primed” position and a second or “dispensed” position. The first slot is positioned distally relative to the second slot, i.e., the first slot is positioned toward the first open end of the syringe body and the second slot is positioned proximally, toward the delivery tip and second open end of the syringe body. In a preferred embodiment, the syringe is provided loaded with medicament in the delivery tip, and having the plunger positioned such that the pawl is engaged with the first slot, i.e., the syringe is provided in a primed position, prior to actuation and deliver of the medicament.

When the plunger is actuated to deliver medicament, the plunger slidingly moves to the second or dispensed position, wherein the pawl engages the second slot. When the pawl engages with the second slot, due to the one-way restriction of movement, the syringe is disabled, and re-use of the syringe is prevented. The syringe is preferably disposed following this single use and delivery of medicament.

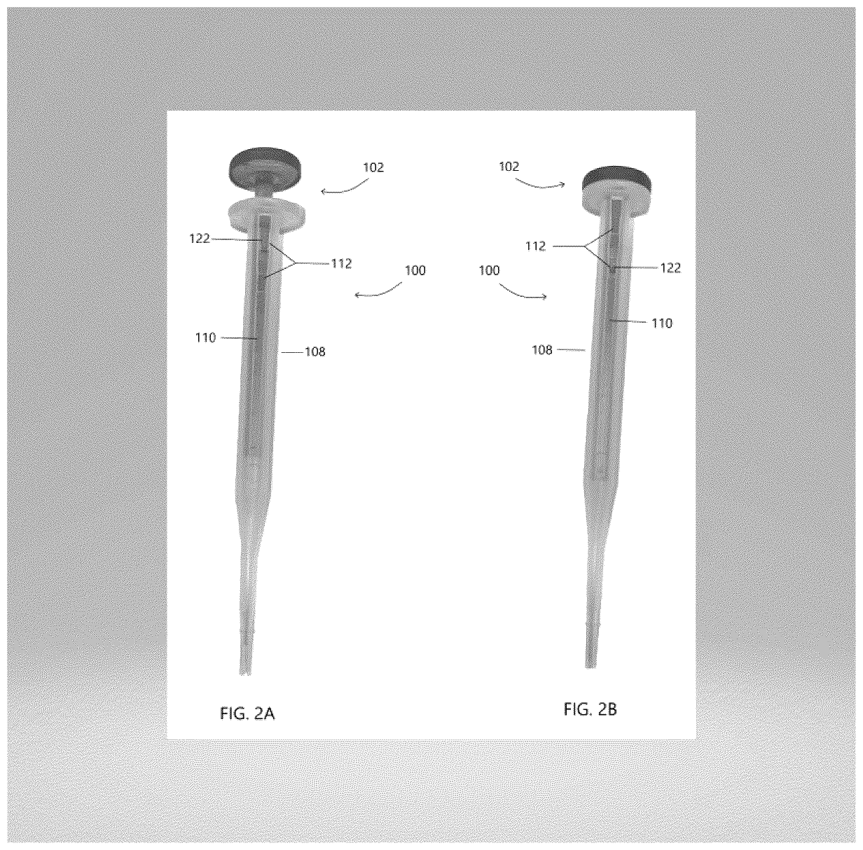

FIG. 2A shows the single-use syringe embodiment of FIG. 1 with the plunger stem 102 housed within the syringe body 100, the plunger tip 120 housed within the delivery tip 114 and lacking a finger flange. The pawl 122 is engaged into a first slot 112 on the sidewall 108 of the syringe body 100. In this position, the syringe can be said to be in a primed or unlocked position.

This contrasts with the syringe of the same embodiment shown in FIG. 2B. The syringe of FIG. 2B shows a fully actuated syringe wherein the pawl 122 of the plunger stem 102 is engaged in the final slot 112 of the sidewall 108. This last slot 112 holds the plunger stem 102 in a locked position and effectively disables the syringe by preventing its reuse. As can be appreciated in FIG. 2B, the first end 104 can also function as a plunger stopper by contacting the plunger stem 102 and preventing it from being expressed further.

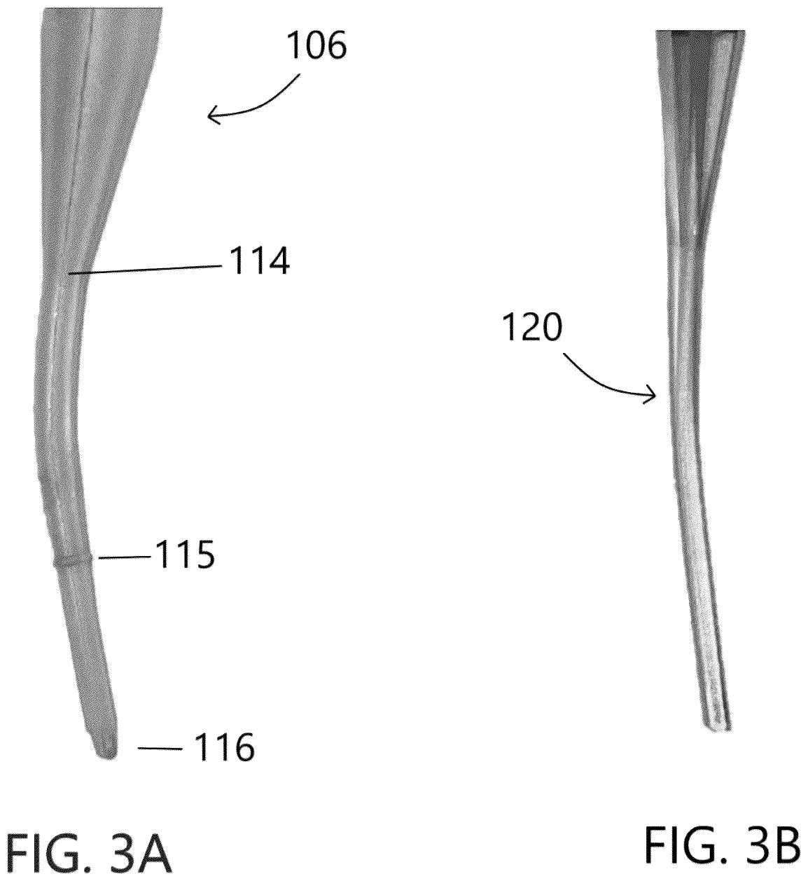

The delivery tip 114 can be better appreciated in FIG. 3A which shows an up-close view of the second end 106 of the syringe embodiment of FIG. 1. The delivery tip 114 shown in FIG. 3A comprises a depth gauge 115 that can also function as a cap stop and an elongated tube having an outer circumference and an internal bore disposed throughout its length. The delivery tip 114 comprises a generally elongate tube, having an internal lumen or bore disposed throughout its entire length (e.g., an opening that extends from one end to the other), preferably round and placed centrally within the tip. On one end of the delivery tip 114 (referred to herein as the “plunger receiving end”), the outer diameter or circumference of the tip can be tapered, forming an elongate, generally cone-shaped tip, wherein an inner diameter is sized to receive the plunger stem 102. The other, opposite end of the tip (referred to herein as the “delivery end”), from which medicament is delivered, has an outer diameter that is smaller or reduced in size compared to the plunger receiving end, and is configured to fit within the periodontal pocket.

The at least one slot can extend partly through or completely through the sidewall such that the inner surface is in communication with the outer surface of the sidewall. To prevent manipulation of the pawl, the area of the syringe sidewall comprising the slots is covered. Preferably, the single-use syringe according to the invention can include a finger flange extending perpendicular to the longitudinal axis of the syringe body to facilitate manipulation of the single-use syringe by a user. In a preferred embodiment, the finger flange is proximate to the first open end of the syringe body and comprises a coupling portion which engages the syringe body and covers the slot areas. More preferably, the coupler and the finger flange are rotationally engaged with the syringe body permitting the finger flange to be rotated about the syringe body as needed by the user.

In a preferred embodiment, the outer aspect or profile of the delivery end of the tip has a tapered outer diameter thereby becoming elliptical in shape, the outer aspect being thereby flattened and consequently reduced in size to advantageously form a two-pronged wedge-shaped end for insertion between a tooth and surrounding gum creating what can be referred to as a “two-pronged bevel” or a “two-pronged beveled delivery port”, thereby allowing the tip to be positioned within the periodontal pocket for delivery of the medicament.

In addition, the delivery segment or delivery end of the tip can be configured such that the end from which medicament is delivered is cut or formed at an acute angle, rather than at a right angle, in relation to the sides of the tip. The delivery end of the tip can alternatively include one or more cutouts or slots. These angled, cut-out- or slotted configurations of the tip delivery end can facilitate exposure of a portion of the medicament disposed in the tip, whereby the tissue contacts the exposed portion of the medicament when delivered and can facilitate secure placement of the medicament in the desired position.

In addition, the tip can be configured or formed wherein the delivery end segment is angled, preferably 90-degrees or less, and more preferably between 10-degrees to about 70-degrees, and most preferably between about 30-degrees and 50-degrees, away from the longitudinal axis of the tip. The angled configuration can be a curved angle to facilitate smooth movement of the plunger within the lumen. Providing an angled tip can be advantageous for inserting the tip into the periodontal pocket for delivery of the medicament. In one preferred embodiment, the tip can be deformable, whereby the user can form the desired angle of the delivery segment of the tip.

In use, the single-use syringe of the invention can preferably be employed to deliver a powdered medicament to a periodontal pocket. The steps of a method of the invention comprise:
a. providing a single-use syringe as described herein;
b. loading a medicament bolus within the delivery tip;
c. positioning the delivery tip of the single-use syringe within a target periodontal pocket of a patient; and
d. actuating the plunger into the locked position to deliver the medicament bolus to the target periodontal pocket.

The method can further comprise disposing of the syringe following use.

The single-use syringe can be provided pre-filled with the medicament bolus. In the pre-filled embodiment, the plunger of the single-use syringe is positioned in a primed position wherein the pawl is engaged with a first slot disposed on the inner surface of the syringe body. When the delivery tip is positioned in the periodontal pocket for administration of the medicament, the plunger is actuated and moved to the second slot disposed on the inner surface of the syringe body, i.e., in a dispensed position, and medicament bolus is delivered to the target periodontal pocket of the patient. The single-use syringe is then disabled and prevented from re-use.

In a further embodiment of the invention, a plurality of single-use syringes is provided in a single package, with written instructions for use. The plurality of syringes permits the user to use a single syringe to administer medicament to the patient, e.g., in a periodontal pocket of the patient, discard the single-use syringe, and then use a second single-use syringe to administer medicament to the patient, e.g., in a second periodontal pocket of the patient, for multiple administrations to multiple periodontal pockets. Typically, a package can comprise a set of four, and up to twelve or more, single-use, disposable syringes pre-loaded with medicament in the delivery tip of each syringe.

In one embodiment of the invention, a plurality of detachable tips can be provided, packaged together, in a tray or other holding case, such that the engagement end of the tip can be fitted onto the open end of the actuator/handle portion without being touched by human hands. This can reduce the chance of contamination of the tip being engaged onto the actuator handle and can reduce the likelihood of cross-contamination of the other tips in the storage tray.

Another embodiment of the subject invention is directed to a method for treating periodontal disease. This method involves providing a single-use syringe as described herein. The single-use syringe can be provided empty or prefilled with a medicament bolus. If provided empty, e.g., the user can load the syringe by pushing medicament bolus into the delivery tip. The user can then position the syringe within the targeted periodontal pocket and actuate the plunger into the locked position to deliver the medicament to the target periodontal pocket.

A further step according to the invention is to dispose of the disabled syringe once it has been fully actuated. The delivery tip can be covered by a delivery tip cap.

1. A disposable, single-use syringe comprising:
a) a hollow syringe body comprising
a sidewall having an outer surface and an inner surface bounding a chamber, the inner surface having disposed thereon at least one slot for matingly receiving and engaging a pawl,
a first open end for receiving a plunger, and
a second open end comprising a delivery tip having a bore therethrough and being configured to hold and deliver a powdered medicament bolus; and
b) a plunger comprising
a plunger stem portion slidably engaging the chamber, said plunger stem portion having a capped distal end and comprising a pawl capable of matingly engaging the at least one slot, and
a plunger delivery portion slidably disposed within the delivery tip and capable of dispensing the medicament bolus from the delivery tip,
wherein, engagement of the pawl and the at least one slot on the inner surface of the sidewall locks the plunger in a dispensed position, thereby disabling the syringe and preventing re-use.

2. The single-use syringe of claim 1, wherein the pawl is integrally formed in the plunger stem portion.

3. The single-use syringe of claim 1, wherein the delivery tip is integrally formed with the syringe body.

4. The single-use syringe of claim 1, wherein the delivery tip is angled between about 10° and about 90° relative to a longitudinal axis of the syringe body.

5. The single-use syringe of claim 1, wherein the delivery tip is angled between about 15° and about 45° relative to a longitudinal axis of the syringe body.

6. The single-use syringe of claim 1, wherein the delivery tip is angled at about 30° relative to a longitudinal axis of the syringe body.

7. The single-use syringe of claim 1, wherein the second open end serves as an exit port for the medicament disposed therein, wherein said exit port is beveled.

8. The single-use syringe of claim 1, wherein the beveled exit port is configured as a two-pronged beveled wedge.

9. The single-use syringe of claim 1 wherein the plunger stem comprises a longitudinal ridge capable of guiding the plunger stem within the syringe chamber and the inner surface of the syringe body sidewall comprises a longitudinal groove configured to matingly engage the longitudinal ridge of the plunger stem.

10. The single-use syringe of claim 1, wherein the inner surface of the syringe body comprises a first slot engaging the pawl in a primed position prior to actuation and deliver of the medicament and a second slot engaging the pawl in a dispensed position following actuation of the plunger and delivery of the medicament bolus, wherein the syringe is disabled and prevents re-use thereof when in the dispensed position.

11. The single-use syringe of claim 1, wherein the delivery tip is an elongate and tapered tube having a cross-sectionally circular internal bore therethrough.

12. The single-use syringe of claim 1, wherein the delivery tip is an elongate tube having a cross-sectionally elliptical outer circumference.

13. The single-use syringe of claim 1, wherein the at least one slot extends through the sidewall such that the inner surface is in communication with the outer surface of the sidewall.

14. The single-use syringe of claim 1, wherein the syringe body further comprises a finger flange proximate to the first open end of the syringe body.

15. The single-use syringe of claim 14, wherein the finger flange is rotationally engaged with the syringe body, allowing the finger flange to rotate about said syringe body.

16. The single-use syringe of claim 15, wherein the finger flange covers the at least one slot.

17. The single-use syringe of claim 1, wherein the delivery tip further comprises a ringed ridge thereon serving as a depth gauge.

18. A method for delivery of a powdered medicament to a periodontal pocket, said method comprising
a. providing a single-use syringe of claim 1;
b. loading a medicament bolus within the delivery tip;
c. positioning the delivery tip of the single-use syringe within a target periodontal pocket of a patient; and
e. actuating the plunger into the locked position to deliver the medicament bolus to the target periodontal pocket.

19. The method of claim 18 further comprising disposing of the syringe following use.

20. The method of claim 18, wherein the single-use syringe is provided pre-filled with the medicament bolus.

21. The method of claim 18, wherein the plunger of the single-use syringe is positioned in a primed position wherein the pawl is engaged with a first slot disposed on the inner surface of the syringe body and said plunger is actuated to be positioned in a second slot disposed on the inner surface of the syringe body to deliver the medicament bolus to the target periodontal pocket of the patient, whereby the single-use syringe is disabled and prevented from re-use.
